(19) United States
(12) Patent Application Publication
(10) Pub. No.: US 2025/0177476 A1
(43) Pub. Date: Jun. 5, 2025

(54) ANG-(1-7) OLIGOPEPTIDE DERIVATIVES FOR USE IN MITIGATING AMYLOID-RELATED IMAGING ABNORMALITIES

(71) Applicant: ProNeurogen, Inc., Tucson, AZ (US)

(72) Inventor: Meredith HAY, Tucson, AZ (US)

(21) Appl. No.: 18/963,930

(22) Filed: Nov. 29, 2024

Related U.S. Application Data
(60) Provisional application No. 63/604,607, filed on Nov. 30, 2023.

(51) Int. Cl. A61K 38/08; A61P 7/04; C07K 7/06

(52) U.S. Cl. CPC A61K 38/085; A61P 7/04; C07K 7/06

(57) ABSTRACT

The present disclosure provides a method of mitigating amyloid-related imaging abnormalities in a subject receiving an antibody therapy, such as a monoclonal antibody, for the treatment of Alzheimer's disease by administering an effective amount of an Angiotensin-(1-7) receptor agonist to the subject.

### SEQUENCE LISTING

The instant application contains a Sequence Listing which has been submitted electronically in XML file format and is hereby incorporated by reference in its entirety. Said XML copy, created on Nov. 27, 2024, is named “51568-003002_Sequence_Listing_11_27_24” and is 11,142 bytes in size.

### BACKGROUND

Amyloid-related imaging abnormalities (ARIA) are abnormalities seen in magnetic resonance imaging (MRI) of the brain of patients diagnosed with Alzheimer's disease (AD). There are two types of ARIA—ARIA-effusion (ARIA-E) and ARIA-hemorrhages (ARIA-H)—and both may be associated with symptoms or completely asymptomatic. The prevalence of ARIA-E and ARIA-H is 6.5% and 7.8% of AD patients, respectively, and asymptomatic ARIA is observed in 80.4% of AD patients.

While ARIA can develop spontaneously as a natural course of AD, most cases of ARIA develop in AD patients undergoing an antibody therapy. Some of the antibodies that have been shown to lead to the development of ARIA include amyloid-modifying monoclonal antibodies such as bapineuzumab, solanezumab, and most recently, aducanumab.

Currently, there are no known treatment options available for ARIA. When ARIA is detected, patients are advised to pause or withdraw from the antibody therapy until the imaging abnormalities and/or associated symptoms are either ameliorated or stabilized, during which AD-associated symptoms may worsen. Accordingly, there is a need for a method of mitigating ARIA in AD patients undergoing antibody therapies for the treatment of AD.

### SUMMARY OF THE INVENTION

In an aspect, the disclosure provides a method for mitigating the effects of amyloid-related imaging abnormalities (ARIA) in a subject receiving an antibody therapy for the treatment of Alzheimer's disease comprising administering a therapeutically effective amount of an Angiotensin-(1-7) (Ang-(1-7)) receptor agonist to the subject. In some embodiments, the antibody therapy comprises administration of one or more antibodies to the subject for the treatment of Alzheimer's disease. In some embodiments, the one or more antibodies comprises a monoclonal antibody. In some embodiments, the one or more antibodies comprises an anti-beta-amyloid antibody. In some embodiments, the one or more antibodies is selected from bapineuzumab, solanezumab, gantenerumab, crenezumab, ponezumab, lecanemab, donanemab, and aducanumab, or a combination thereof.

In some embodiments, the Ang-(1-7) receptor agonist comprises an Ang-(1-7) oligopeptide derivative. In some embodiments, the Ang-(1-7) oligopeptide derivative has the formula: A¹-A²-A³-A⁴. A⁵-A⁶-A⁷-A⁸ (SEQ ID NO: 1) wherein: A¹ is selected from the group consisting of aspartic acid, glutamic acid, alanine, and glycosylated forms thereof; A² is selected from the group consisting of arginine, histidine, lysine, and glycosylated forms thereof; A³ is selected from the group consisting of valine, alanine, isoleucine, leucine, and glycosylated forms thereof; A⁴ is selected from the group consisting of tyrosine, phenylalanine, tryptophan, and glycosylated forms thereof; A⁵ is selected from the group consisting of isoleucine, valine, alanine, leucine, and glycosylated forms thereof; A⁶ is selected from the group consisting of histidine, arginine, lysine, and glycosylated forms thereof; A⁷ is selected from the group consisting of proline, glycine, serine, and glycosylated forms thereof; and A⁸ can be present or absent, wherein when A⁸ is present, A⁸ is selected from the group consisting of serine, threonine, hydroxyproline, and glycosylated forms thereof.

In some embodiments, at least one of A¹-A⁸ is glycosylated with a monosaccharide or disaccharide. In some embodiments, A⁸ is glycosylated with a monosaccharide or disaccharide or A⁸ is absent and A⁷ is glycosylated with a monosaccharide or disaccharide. In some embodiments, at least one of the monosacharides or disaccharides is selected from the group consisting of glucose, galactose, xylose, fucose, rhamnose, lactose, cellobiose, and melibiose. In some embodiments, (a) A⁷ is a serine or a glycosylated form thereof and A⁸ is absent or (b) A⁸ is serine or a glycosylated form thereof. In some embodiments, (a) A⁷ is glycosylated with glucose or lactose and A⁸ is absent or (b) A⁸ is glycosylated with glucose or lactose. In some embodiments, (a) A⁷ is terminated with an amino group and A⁸ is absent or (b) A⁸ is terminated with an amino group.

In some embodiments, the Ang-(1-7) oligopeptide derivative is selected from the group consisting of any one of SEQ ID NOs: 6-13. In some embodiments, the Ang-(1-7) oligopeptide derivative is SEQ ID NO: 6. In some embodiments, the Ang-(1-7) oligopeptide derivative is SEQ ID NO: 7. In some embodiments, the Ang-(1-7) oligopeptide derivative is SEQ ID NO: 8. In some embodiments, the Ang-(1-7) oligopeptide derivative is SEQ ID NO: 9. In some embodiments, the Ang-(1-7) oligopeptide derivative is SEQ ID NO: 10. In some embodiments, the Ang-(1-7) oligopeptide derivative is SEQ ID NO: 11. In some embodiments, the Ang-(1-7) oligopeptide derivative is SEQ ID

NO: 12. In some embodiments, the Ang-(1-7) oligopeptide derivative is SEQ ID NO: 13. In some embodiments, the Ang-(1-7) oligopeptide derivative comprises at least one D-amino acid. In some embodiments, each amino acid is a D-amino acid.

In some embodiments, the Ang-(1-7) oligopeptide derivative is formulated as an injectable solution or dispersion. In some embodiments, the Ang-(1-7) oligopeptide derivative is administered to the subject intravenously, subcutaneously, intramuscularly, intraperitoneally, intracerebroventricularly, or by intrathecal injection.

In some embodiments, the Ang-(1-7) oligopeptide derivative is administered to the subject in an amount of 0.1 mg/day to 1000 mg/day (e.g., 0.1 mg/day to 800 mg/day, 0.1 mg/day to 600 mg/day, 0.1 mg/day to 400 mg/day, 0.1 mg/day to 200 mg/day, 0.1 mg/day to 100 mg/day, 0.1 mg/day to 50 mg/day, 0.1 mg/day to 10 mg/day, 10 mg/day to 1000 mg/day, 100 mg/day to 1000 mg/day, 200 mg/day to 1000 mg/day, 400 mg/day to 1000 mg/day, 600 mg/day to 1000 mg/day, 800 mg/day to 1000 mg/day, 1 mg/day to 50 mg/day, or 10 mg/day to 100 mg/day). In some embodiments, the Ang-(1-7) oligopeptide derivative is administered to the subject in an amount of 0.1-50 mg/kg (e.g., 0.1-40 mg/kg, 0.1-30 mg/kg, 0.1-20 mg/kg, 0.1-10 mg/kg, 0.1-1 mg/kg, 1-50 mg/kg, 5-50 mg/kg, 10-50 mg/kg, 20-50 mg/kg, 30-50 mg/kg, 40-50 mg/kg, 1-20 mg/kg, or 5-10 mg/kg) of the subject's body weight per day.

In some embodiments, the Ang-(1-7) oligopeptide derivative is administered to the subject 1 to 4 (e.g., 1, 2, 3, or 4) times daily. In some embodiments, the Ang-(1-7) oligopeptide derivative is administered to the subject once a day.

In some embodiments, the Ang-(1-7) oligopeptide derivative is formulated as an extended-release injectable gel. In some embodiments, the extended-release injectable gel formulation is administered to the subject intravenously, subcutaneously, intramuscularly, intraperitoneally, intracerebroventricularly, or by intrathecal injection.

In some embodiments, the extended-release injectable gel formulation is administered to the subject at least once a year. In some embodiments, the extended-release injectable gel formulation is administered to the subject from once a 1 week to once a year. In some embodiments, the extended-release injectable gel formulation is administered to the subject from once a 1 week to once a month.

In some embodiments, the Ang-(1-7) oligopeptide derivative is administered to the subject in amount of 10-14 mg/day, 70 mg/week, 140 mg/two weeks, or 280 mg/month. In some embodiments, the extended-release injectable gel formulation is administered to the subject through a needle having a diameter of 20 to 25 gauge (e.g., 20, 21, 22, 23, 24, or 25 gauge).

In some embodiments, the Ang-(1-7) oligopeptide derivative is administered to the subject from 1 day to 6 months (e.g., 1 day to 4 months, 1 day to 2 months, 1 day to 1 month, 1 day to 5months, 1 day to 1 month, 2 weeks to 6 months, 1 month to 6 months, 2 months to 6 months, 3 months to 6 months, 4 months to 6 months, 5 months to 6 months, 1 week to 2 weeks, 1 week to 1 month, or 2 weeks to 2 months) prior to when the antibody therapy for the treatment of Alzheimer's disease is first administered to the subject. In some embodiments, the Ang-(1-7) oligopeptide derivative is administered to the subject from 1 week to 3 months (e.g., 1 week to 10 weeks, 1 week to 8 weeks, 1 week to 6 weeks, 1 week to 4 weeks, 1 week to 2 weeks, 2 weeks to 12 weeks, 4 weeks to 12 weeks, 6 weeks to 12 weeks, 8 weeks to 12 weeks, 10 weeks to 12 weeks, 2 weeks to 8 weeks, or 3 weeks to 6 weeks) prior to when the antibody therapy for the treatment of Alzheimer's disease is first administered to the subject. In some embodiments, the Ang-(1-7) oligopeptide derivative is administered to the subject from 1 week to 2 months (e.g., 1 week to 8 weeks, 1 week to 7 weeks, 1 week to 6 weeks, 1 week to 5 weeks, 1 week to 4 weeks, 1 week to 3 weeks, 1 week to 2 weeks, 2 weeks to 8 weeks, 3 weeks to 8 weeks, 4 weeks to 8 weeks, 5 weeks to 8 weeks, 6 weeks to 8 weeks, 7 weeks to 8 weeks, 2 weeks to 5 weeks, or 4 weeks to 6 weeks) prior to when the antibody therapy for the treatment of Alzheimer's disease is first administered to the subject. In some embodiments, the Ang-(1-7) oligopeptide derivative is administered to the subject for at least 1 month.

In some embodiments, the Ang-(1-7) oligopeptide derivative is administered to the subject for a period of from about 1 month to about 2 years (e.g., 1 month to 18 months, 1 month to 12 months, 1 month to 6 months, 1 month to 2 months, 2 months to 24 months, 6 months to 24 months, 12 months to 24 months, 18 months to 24 months, 4 months to 12 months, or 6 months to 18 months). In some embodiments, the Ang-(1-7) oligopeptide derivative is administered to the subject for a period of from about 1 month to about 1 year (e.g., 1 month to 11 months, 1 month to 10 months, 1 month to 9 months, 1 month to 8 months, 1 month to 7 months, 1 month to 6 months, 1 month to 5 months, 1 month to 4 months, 1 month to 3 months, 1 month to 2 months, 2 months to 12 months, 3 months to 12 months, 4 months to 12 months, 5 months to 12 months, 6 months to 12 months, 7 months to 12 months, 8 months to 12 months, 9 months to 12 months, 10 months to 12 months, 11 months to 12 months, 4 months to 8 months, or 2 months to 8 months).

In some embodiments, the subject is a human subject. In some embodiments, the subject has been diagnosed as having Alzheimer's disease.

### BRIEF DESCRIPTION OF THE DRAWINGS

FIG. 1A is an image showing the microbleed (ARIA-h) in the brain of a mouse.

FIG. 1B is a graph measuring the number of cerebral microbleeds in a given area for mice that were administered (1) the PNA⁵ vehicle, without PNA^{5,} and an anti-Aβ antibody; (2) PNA⁵ and an anti-Aβ antibody; (3) the PNA⁵ vehicle, without PNA⁵ and IgG as a control; or (4) PNA⁵ and IgG.

FIG. 2 is a graph showing the Nest building score in mice after they were administered: (1) the PNA⁵ vehicle, without PNA^{5,} and PBS, (2) PNA⁵ and PBS; (3) vehicle and an anti-Aβ antibody; or (4) PNA⁵ and an anti-Aβ antibody.

### DEFINITIONS

The term “administration” or “administering” refers to a method of giving a dosage of a pharmaceutical composition to a patient, where the method is systemic, e.g., oral, topical, transdermal, by inhalation, intravenous, intraperitoneal, intracerebroventricular, intrathecal, or intramuscular. The preferred method of administration can vary depending on various factors, e.g., the components of the pharmaceutical composition, site of administration, and severity of the symptoms being treated.

As used herein, the term “Alzheimer's disease” or “AD” refers to a neurodegenerative disease and the most common cause of dementia. This disease manifests as a gradual but progressive decline in memory, thinking skills and behavior that is accelerated relative to normal aging (Reitz et al. 2011 Nat Rev Neurol 7:137-152). Eventually, patients are unable to recognize familiar people or carry out the simplest task. Alzheimer's disease is, at this time, among the leading causes of death in the

United States (US). There are two predominant forms of the disease: Familial Alzheimer's disease is typically caused by dominant genetic mutations. This form of the disease is a rare and devastating illness with onset occurring in mid-life. The second and far more common form of the disease is Sporadic or Late onset Alzheimer's disease.

As used herein, the term “Ang-(1-7) oligopeptide derivative” refers to an oligopeptide in which one or more amino acid residue is either modified or different than the amino acid residue of the corresponding native Ang-(1-7). The term “Ang-(1-7) oligopeptide derivative” also includes an oligopeptide of eight amino acid residues as discussed in more detail below.

As used herein, the terms “approximately” or “about” in reference to a number are generally taken to include numbers that fall within a range of 5%, 10%, 15%, or 20% in either direction (greater than or less than) of the number, unless otherwise stated or otherwise evident from the context (except where such number would be less than 0% or exceed 100% of a possible value).

As used herein, the term “average rate of release” refers to the numerical average of the rate of drug release from a formulation of the disclosure over a defined period of time. According to the present disclosure, the average rate of release may be quantified as:

$$\frac{{\sum }_{0}^{n} \frac{\% of total drug released}{time interval}}{n}$$

where n=total number of time intervals.

As used herein, the term “burst release” refers to a physical property of controlled-release drug formulations characterized by a large initial release of the therapeutically active drug cargo following placement of the formulation in a release medium. Burst release results in a higher initial drug dose and may also reduce the effective lifetime of a formulation by prematurely depleting levels of the therapeutic drug upon administration. Generally, burst release may be measured from the initial time (t₀) of placement of the formulation in a release medium or administration to a subject to t₀+Δt, where Δt is a time interval ranging from 1 to 24 hours.

As used herein, the term “carbohydrate” refers to pentose and hexose of empirical formula (CH₂O)ₙ, where n is 5 for pentose and 6 for hexose. A carbohydrate can be monosaccharide, disaccharide, oligosaccharide (e.g., 3-20, typically 3-10, and often 3-5 monomeric saccharides are linked together), or polysaccharide (e.g., greater than 20 monomeric saccharide units). The term carbohydrate may refer specifically to a monosaccharide and/or disaccharide. However, it should be appreciated that the scope of the invention is not limited to mono-or disaccharides. The terms “carbohydrate” and “saccharide” are used interchangeably herein.

As used herein, the term “combinations thereof,” references to any modifications (e.g., carbohydrate modifications) of Ang-(1-7) oligopeptide derivatives, refers to oligopeptides in which two, three, four, five, six, seven, or eight of the individual amino acids are modified by the attachment of a carbohydrate. For Ang-(1-7) oligopeptide derivatives having a plurality of carbohydrate modifications, the modifying carbohydrates may be the same on every modified amino acid, or the several modified amino acids may comprise a mixture of different carbohydrates.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties
of naturally-occurring amino acids

| | | | | Electrostatic | |
|---|---|---|---|---|---|
| | 3 | | Side- | character at | |
| | Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic | Asp | D | polar | anionic | intermediate |
| acid | | | | | |
| Cysteine | Cys | C | nonpolar | neutral | intermediate |
| Glutamic | Glu | E | polar | anionic | intermediate |
| acid | | | | | |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and > 200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “derivative” refers to any chemical modification of the amino acid, such as alkylation (e.g., methylation or ethylation) of the amino group or the functional group on the side chain, removal of the side-chain functional group, addition of a functional group (e.g., hydroxyl group on proline), attachment of mono-or di-carbohydrate (e.g., via glycosylation), etc. Exemplary glycosylated derivatives include hydroxyl group on serine that is glycosylated with glucose, galactose, ribose, arabinose, xylose, lyxose, allose, altrose, mannose, gulose, iodose, talose, fucose, rhamnose, etc., as well as disaccharides and amino sugars such as galactosamine, glucosamine, sialic acid, N-acetyl glucosamine, etc. Amino acid derivatives also include modified or unmodified D-amino acids.

As used herein, the terms “effective amount,” “therapeutically effective amount,” and “sufficient amount” of a formulation or composition described herein refer to a quantity sufficient to, when administered to the subject effect beneficial or desired results, including clinical results, and, as such, an “effective amount” or synonym thereto depends upon the context in which it is being applied. For example, in the context of treating a cognitive impairment (e.g., vascular dementia), it is an amount of the formulation or composition sufficient to achieve a treatment response as compared to the response obtained without administration of the formulation or composition. The amount of a given formulation or composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art by routine methods known in the art. Dosage regimen may be adjusted to provide the optimum therapeutic response.

As used herein, the term “extended-release” refers to a physical property of a pharmaceutical formulation of the disclosure such that the formulation is configured to gradually release a steady amount of a biologically-active therapeutic agent dispersed therein over a defined period of time (e.g., over a period of a day, week, month, etc.). Extended-release formulation offers benefits for the delivery of therapeutic agents to patients by allowing the formulation to provide continuous dosing of the therapeutic agent in the subject without requiring frequent administration. Thus, extended-release formulations mitigate the problems associated with delivering a high dose of a drug to a patient and improve patient compliance. According to the present disclosure, extended-release can be defined as a percent (%) release of a total dose of a therapeutic agent (e.g., oligopeptides of the disclosure) contained in an extended-release formulation over a defined time interval. Furthermore, extended-release may also require that the rate of release of a drug from a formulation of the disclosure does not exceed a specific rate of release within a defined period of time (e.g., rate of drug release does not exceed, e.g., X %/Y hours).

As used herein, the term “inverso modified” refers to a peptide which is made up of D-amino acids in which the amino acid residues are assembled in the same direction as the native peptide with respect to which it is inverso modified.

As used herein, the terms “mitigate” and “mitigating” refer to reducing the severity of or number of occurrences of one or more symptoms of that present with ARIA. Symptoms that present with ARIA which may be mitigated include but are not limited to reducing edema, effusion, microhemorrhage, superficial siderosis, headache, confusion, visual disturbances, visuospatial impairment, praxis difficulties, and neuropsychiatric symptoms is reduced in severity in comparison to the performance of the subject prior to receiving the treatment.

As used herein, the term “native” refers to any sequence of L amino acids used as a starting sequence or a reference for the preparation of partial or complete retro, inverso, or retro-inverso analogues. Thus, the term “native Ang-(1-7)” refers to an oligopeptide having the same amino acid sequence as that of endogenous Ang-(1-7). It should be appreciated that the use of the term “native” does not imply naturally-occurring, although it can include the endogenous form of Ang-(1-7). The term “native” merely refers to having the same amino acid sequence as that of endogenous Ang-(1-7) without any modification of the amino acid residues. Accordingly, the term “native Ang-(1-7)” includes both synthetic Ang-(1-7) and endogenous Ang-(1-7) as long as the amino acid residues are the same and are not modified.

As used herein, the term “oligopeptide” refers to an amino acid chain of any length, but typically amino acid chain of about fifteen or less amino acids, ten or less amino acids, eight or less amino acids, or seven or eight amino acids. An exemplary oligopeptide includes the Ang-(1-7) heptapeptide or a variant thereof (e.g., any one of SEQ ID NOs: 6-13). It should be appreciated that one or more of the amino acids of Ang-(1-7) can be replaced with an “equivalent amino acid”, e.g., L (leucine) can be replaced with isoleucine or other hydrophobic side-chain amino acid such as alanine, valine, methionine, etc., and amino acids with polar uncharged side chain can be replaced with other polar uncharged side chain amino acids. While Ang-(1-7) comprises 7 amino acids, in some embodiments the oligopeptide derivative of the invention has eight or less amino acids.

As used herein, the term “percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, e.g., using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)
   where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject in order to prevent, treat, or control a particular disease or condition affecting or that may affect the subject.

As used herein, the term “pharmaceutically acceptable” refers to those compounds, materials, compositions, and/or dosage forms, which are suitable for contact with the tissues of a subject without excessive toxicity, irritation, allergic response, and other problem complications commensurate with a reasonable benefit/risk ratio.

As used herein, the term “prevent,” when used in connection with the occurrence of a disease, disorder, and/or condition, refers to reducing the risk of developing the disease, disorder, and/or condition for which the subject is at risk of developing.

As used herein, the terms “poly-glycolic acid,” “polyglycolide,” and “PGA” refer to a polymeric form of glycolic acid of variable lengths and weights. PGA is a biodegradable, thermoplastic polymer that is commonly used in medicinal suture and drug delivery systems. PGA can be incorporated into a polymer matrix containing poly-lactic acid (PLA) to produce poly-(lactic co-glycolic acid) (PLGA).

As used herein, the terms “poly-lactic acid,” “polylactide,” and “PLA” refer to a thermoplastic polyester containing a variable number of lactic acid monomers. PLA has been employed in a number of commercially available products, including medical implants, drug delivery systems, and decomposable materials. PLA can be incorporated into a polymer matrix containing PGA, i.e., PLGA.

As used herein, the terms “poly-(lactic co-glycolic acid)” or “PLGA” refer to a copolymer containing PGA and PLA at various ratios. PLGA can be synthesized as a random or block copolymer to impart specific properties. Generally, higher PGA content in the PLGA copolymer will result in improved hydrolysis of PLGA. PLGA or monomeric components thereof may be end-capped with esters (ester-capped) or with a free carboxylic acid (acid-capped). PLGA is commonly used as a drug delivery vehicle owing to its biodegradability and tolerability. For example, an FDA approved Lupron depot uses PLGA as a drug vehicle for the treatment of advanced prostate cancer. As used herein, the term “retro modified” refers to a peptide which is made up of L-amino acids in which the amino acid residues are assembled in the opposite direction to the native peptide with respect to which it is retro modified. As used herein, the term “retro-inverso modified” refers to a peptide which is made up of D-amino acids in which the amino acid residues are assembled in the opposite direction to the native peptide with respect to which it is retro-inverso modified. Thus, native Ang-(1-7) (L-amino acids, N→C direction) is: Asp-Arg-Val-Tyr-Ile-His-Pro, i.e., DRVYIHP (SEQ ID NO: 2). Retro-inverso Ang-(1-7) (D-amino acids, C→N direction) is: DRVYIHP (SEQ ID NO: 3). Retro Ang-(1-7) (L-amino acids, C→N direction) is: DRVYIHP (SEQ ID NO: 4). Finally, inverso Ang-(1-7) (D-amino acids, N→C direction) is: DRVYIHP (SEQ ID NO: 5). The use of D-amino acids in the context of inverso modified and retro-inverso modified Ang-(1-7) oligopeptide derivatives is not intended to be limiting on the use of D-amino acids in the oligopeptides. As discussed in more detail below, fewer than all of the amino acids in an Ang-(1-7) oligopeptide derivative may be D-amino acids.

As used herein, the terms “sucrose acetoisobutyrate” and “SAIB” refer to an additive used for various purposes, including additives in beverages, color cosmetics and skin care, flavorings, fragrances, hair products, and horse styling products. Within the context of the present invention, SAIB is used as a biocompatible polymer suitable for producing extended-release gel formulations for sustained release of one or more of the therapeutic peptides disclosed herein (e.g., Ang-(1-7)). SAIB has the chemical structure shown below.

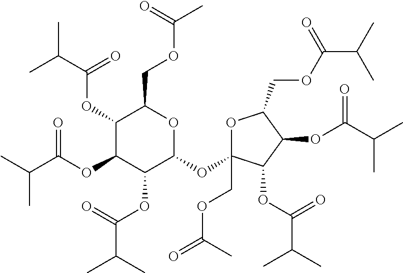 [SMILES: CC(=O)OC[C@H]1O[C@H](O[C@]2(COC(C)=O)O[C@H](COC(=O)C(C)C)[C@@H](OC(=O)C(C)C)[C@@H]2OC(=O)C(C)C)[C@H](OC(=O)C(C)C)[C@@H](OC(=O)C(C)C)[C@@H]1OC(=O)C(C)C]

As used herein, the terms “subject” and “patient” refer to a mammal (e.g., human). A subject to be treated according to the methods described herein may be one who has been diagnosed with Alzheimer's disease. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. As used herein, the terms “treatment” and “treating,” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “therapeutically effective amount” of a formulation or composition described herein refers to a quantity sufficient to, when administered to the subject effect beneficial or desired results, including clinical results, and, as such, an “effective amount” or synonym thereto depends upon the context in which it is being applied. The “therapeutically effective amount” will vary depending on various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art by routine methods known in the art. Dosage regimen may be adjusted to provide the optimum therapeutic response.

### DETAILED DESCRIPTION

The claimed invention is directed to a method of mitigating amyloid-related imaging abnormalities (ARIA) in a subject receiving an antibody therapy for Alzheimer's disease (AD), wherein the method comprises the administration of an effective amount of an Angiotensin-(1-7) (Ang-(1-7)) receptor agonist. AD patients undergoing antibody therapies for AD, such as an amyloid-modifying monoclonal antibody therapy, are susceptible to the development of ARIA, for which there currently is no therapeutic option. Angiotensin-(1-7) oligopeptide derivatives are modified native Ang-(1-7) with enhanced endothelial interaction and penetration into the brain. Pretreatment with Ang-(1-7) oligopeptide derivatives and/or concomitant administration of antibody therapies and Ang-(1-7) oligopeptide derivatives may provide therapeutic benefits to AD patients.

Ang-(1-7) Oligopeptide Derivatives

Ang-(1-7) oligopeptide derivatives refer to oligopeptides whose amino acid sequence of any one or more of Ang-(1-7) is modified (e.g., via methylation, presence of a functional group, such as hydroxy group on proline), attached to a carbohydrate, is replaced with corresponding D-amino acid or an “equivalent amino acid” as defined above, and/or the terminal amino group end or the carboxyl end of Ang-(1-7) is modified. For example, the carboxylic acid end can be modified to be an amide, an amine, a thiol, or an alcohol functional group, or one in which an additional amino acid residue is present compared to native Ang-(1-7). It should be appreciated that the term “Ang-(1-7) oligopeptide derivative” excludes the native Ang-(1-7), i.e., amino acid sequences of endogenous Ang-(1-7) without any modification.

In some embodiments, oligopeptides of the invention have the amino group on the carboxylic acid terminal end (i.e., the-OH group of the carboxylic acid is replaced with —NR^{a}R^{b}, where each of R^{a} and R^{b} is independently hydrogen or Ci-C₆ alkyl) and/or have one or more amino acid residues that are (i) replaced with a corresponding D-amino acid, (ii) glycosylated, (iii) replaced with another amino acid, (iv) or a combination thereof.

Still in other embodiments, the oligopeptide derivative of the invention is retro-inverso Ang-(1-7). Yet in other embodiments, the oligopeptide derivative of the invention is retro Ang-(1-7). In other embodiments, the oligopeptide derivative of the invention is inverso Ang-(1-7).

Other embodiments of the invention include Ang-(1-7) oligopeptide derivatives in which at least one or more, typically one or two, and often only one amino acid is attached to a carbohydrate. Generally, the carbohydrate is attached to the amino acid via glycosylation. Typically, the carbohydrate is a mono-or di-carbohydrate. Exemplary mono-and di-carbohydrates that can be used in the invention include, but are not limited to, xylose, fucose, rhamnose, glucose, lactose, cellobiose, melibiose, and a combination thereof.

In one particular embodiment, the oligopeptide that is administered to the subject is Ang-(1-7) oligopeptide derivative of the formula:

(SEQ ID NO: 1)
A¹-A²-A³-A⁴-A⁵-A⁶-A⁷-A⁸,

where A¹ is selected from the group consisting of aspartic acid, glutamic acid, alanine, and glycosylated forms thereof; A² is selected from the group consisting of arginine, histidine, lysine, and glycosylated forms thereof; A³ is selected from the group consisting of valine, alanine, isoleucine, leucine, and glycosylated forms thereof; A⁴ is selected from the group consisting of tyrosine, phenylalanine, tryptophan, and glycosylated forms thereof; A⁵ is selected from the group consisting of isoleucine, valine, alanine, leucine, and glycosylated forms thereof; A⁶ is selected from the group consisting of histidine, arginine, lysine, and glycosylated forms thereof; A⁷ is selected from the group consisting of proline, glycine, serine, and glycosylated forms thereof; and A⁸ can be present or absent, wherein when A⁸ is present, A⁸ is selected from the group consisting of serine, threonine, hydroxyproline, and glycosylated forms thereof, provided (i) at least one of A¹-A⁸ is optionally substituted with a mono-or di-carbohydrate; or (ii) when A⁸ is absent: (a) at least one of A¹-A⁷ is substituted with a mono-or di-carbohydrate, (b) A⁷ is terminated with an amino group, or (c) a combination thereof.

In some embodiments, A¹ is the amino terminal end of the oligopeptide and A⁸ (or A⁷ when A⁸ is absent) is the carboxyl terminal end. Still in other embodiments, A¹ is the carboxyl terminal end and A⁸ (or A⁷ when A⁸ is absent) is the amino terminal end. Yet in other embodiments, the carboxylic acid functional group of the carboxyl terminal end is modified as an amide functional group, an amine functional group, a hydroxyl functional group, or a thiol functional group. The amide and the amine functional groups can be non-alkylate, mono-alkylated or di-alkylated.

Yet in other embodiments, the carbohydrate comprises glucose, galactose, xylose, fucose, rhamnose, or a combination thereof. In some instances, the carbohydrate is a mono-carbohydrate, whereas in other instances, the carbohydrate is a di-carbohydrate.

In other embodiments, at least one of A¹-A⁸ is substituted with a mono-carbohydrate. Still in other embodiments, at least one of A¹-A⁸ is substituted with a di-carbohydrate. It should be appreciated that the scope of the invention also includes those oligopeptides having both mono-and di-carbohydrates.

Exemplary di-carbohydrates that can be used in oligopeptides of the invention include, but are not limited to, lactose, cellobiose, melibiose, and a combination thereof. However, it should be appreciated that the scope of the invention includes oligopeptides that are substituted with any di-carbohydrates known to one skilled in the art.

In one particular embodiment, A⁸ is serine or glycosylated forms thereof. In some instances, the carboxylic acid moiety of the serine is modified as an amide or an amine. In one case, serine is terminated as an amino group. Still in other embodiments, the serine residue of A⁸ is glycosylated with glucose or lactose.

Yet in other embodiments, at least one, typically at least two, generally at least three, often at least four, still more often at least five, yet still more often at least six, and most often all of A¹-A⁸ is D-amino acid.

In particular, in some specific embodiments, said oligopeptide is retro modified, inverso modified, or retro-inverso modified.

Another aspect of the invention provides oligopeptides, such as Ang-(1-7) oligopeptide derivatives, having eight amino acids or less, typically seven or eight amino acid residues. In some embodiments, one or more amino acids have attached thereto a carbohydrate group. Often the carbohydrate group is attached to the oligopeptide via glycosylation. The carbohydrate can be attached to the oligopeptide via any of the side chain functional group of the amino acid or the amide group. Accordingly, the scope of the invention includes, but is not limited to, O-glycosylate, N-glycosylate, S-glycosylated oligopeptides. The term “X-glycosylated” refers to having a carbohydrate attached to the oligopeptide via the heteroatom “X” of the amino acid. For example, for serine whose side-chain functional group is hydroxyl, “O-glycosylated” means the carbohydrate is attached to the serine's side-chain functional group, i.e., the hydroxyl group. Similarly, “N-glycosylation” of leucine refers to having the carbohydrate attached to the amino side-chain functional group of leucine. Typically, the glycosylation is on the side-chain functional group of the amino acid. In some embodiments, the Ang-(1-7) oligopeptide derivative is glycosylated with xylose, fucose, rhamnose, glucose, lactose, cellobiose, melibiose, or a combination thereof.

Yet in other embodiments, the carboxylic acid terminal end of said glycosylated Ang-(1-7) oligopeptide derivative is substituted with an amino group. When referring to the carboxyl acid terminal end being substituted with an amino group, it means-OH group of the carboxylic acid is replaced with —NH₂ group. Thus, the actual terminal end functional group is an amide, i.e., rather than having the oligopeptide being terminated at the carboxylic acid terminal end with a functional group —CO₂H, the carboxylic acid terminal end is terminated with an amide group (i.e., —CO₂NR′₂, where each R′ is independently hydrogen or C₁-C₁₂ alkyl). Still in other embodiments, the carboxylic acid terminal group is terminated with a hydroxyl or a thiol group. In some embodiments, the modified carboxylic acid terminal group is used to attach the carbohydrate, e.g., via glycosylation.

One of the purposes of the invention was to produce Ang-(1-7) oligopeptide derivatives to enhance efficacy of action, in vivo stabilization, and/or penetration of the blood-brain barrier. Improved penetration of the blood-brain barrier facilitates cerebral entry of the Ang-(1-7) oligopeptide derivatives of the invention, and, consequently, intrinsic-efficacy. To improve (i.e., increase) penetration of the blood-brain barrier, in some embodiments the Ang-(1-7) oligopeptide derivative is attached to at least one mono-or di-carbohydrate.

Without being bound by any theory, it is believed that the oligopeptides described herein that are glycosylated exploit the inherent amphipathicity of the folded Ang-(1-7) glycopeptides (i.e., glycosylated oligopeptides of the invention) and the “biousian approach” to deliver the glycosylated oligopeptides of the invention across the blood-brain barrier. In some instances, the amount of increase in crossing the blood-brain barrier by oligopeptides of the invention is at least 6%, typically at least 10%, and often at least 15% compared to native Ang-(1-7). In other instances, oligopeptides of the invention have in vivo half-life of at least 30 min, typically at least 40 min, and often at least 50 min. Alternatively, compared to native Ang-(1-7), oligopeptides of the invention exhibit at least 50-fold, typically at least 75 fold, and often at least 100 fold increase in in vivo half-life.

In other embodiments, oligopeptides described herein exhibit enhanced vascular efficacy. Without being bound by any theory, it is generally recognized that blood-brain barrier transport occurs via an absorptive endocytosis process on the blood side of the endothelium of the brain capillaries followed by exocytosis on the brain side, leading to overall transcytosis. It is also believed that for this process to be efficient, the oligopeptide must bind to the membrane for some period of time and must also be able to exist in the aqueous state for some period of time (biousian nature). It is believed that effective drug delivery and blood-brain barrier transport requires a biousian glycopeptide that has at least two states: (1) a state defined by one or more membrane-bound conformations that permit or promote endocytosis; and (2) a state defined by a water-soluble, or random coil state that permits “membrane hopping” and, presumably, vascular efficacy.

In general, the degree of glycosylation does not have a large effect on the structure of the individual microstates. Thus, altering the degree of glycosylation allows for the modulation of aqueous vs. membrane-bound state population densities without significantly affecting the overall structure of the oligopeptide. Moreover, it is believed that glycosylation also promotes stability to peptidases, thereby increasing the half-life of the Ang-(1-7) oligopeptide derivatives in vivo.

TABLE 2
Exemplary Ang-(1-7) oligopeptide derivatives.

| ANG-(1-7) | Amino acid residue | | | | | | | | Carboxyl terminal |
|---|---|---|---|---|---|---|---|---|---|
| Oligopeptide | 1 | 2 | 3 | 4 | 5 | 6 | 7 | 8 | end functional group |
| Native ANG-(1-7) | Asp | Arg | Val | Tyr | Ile | His | Pro | — | OH (SEQ ID NO: 2) |
| ANG-(1-7)_V1 | Asp | Arg | Val | Tyr | Ile | His | Pro | — | NH₂ (SEQ ID NO: 6) |
| ANG-(1-7)_V2 | Asp | Arg | Val | Tyr | Ile | His | Pro | Ser° | NH₂ (SEQ ID NO: 7) |
| ANG-(1-7)_V3 | Asp | Arg | Val | Tyr | Ile | His | Pro | Ser* | NH₂ (SEQ ID NO: 8) |
| ANG-(1-7)_V4 | Asp | Arg | Val | Tyr | Ile | His | Pro | Ser** | NH₂ (SEQ ID NO: 9) |
| ANG-(1-7)_V5 | Asp | Arg | Val | Tyr | Ile | His | Ser* | — | NH₂ (SEQ ID NO: 10) |
| (PNA5) | | | | | | | | | |
| ANG-(1-7)_V6 | Ala | Xxx | Yyy | Tyr | Ile | Zzz | Pro | Ser°*/** | NH₂ (SEQ ID NO: 11) |
| ANG-(1-7)_V7 | Asp | Arg | Xxx | Tyr | Yyy | His | Pro | Ser°*/** | NH₂ (SEQ ID NO: 12) |
| ANG-(1-7)_V8 | Asp | Arg | Xxx | Zzz | Yyy | His | Pro | Ser°*/** | NH₂ (SEQ ID NO: 13) |

Table 2 above shows some of the representative oligopeptides of the invention. In particular, these oligopeptides can be considered Ang-(1-7) oligopeptide derivatives. In Table 2, Vn (e.g., V1, V2, V3, etc.) represents an Ang-(1-7) oligopeptide variant identifier (e.g., V1 is Ang-(1-7) variant oligopeptide 1), Ser^{o} refers to an unglycosylated serine residue, Ser* refers to a glycosylated serine residue, and Ser** refers to a lactosylated serine residue, Xxx, Yyy, and Zzz refer to any amino acid residue. As shown in Table 2, some of the oligopeptides have carbohydrate attached to the native Ang-(1-7) peptide. These peptides are sometimes referred to as glycopeptides. Studies have shown that inherent binding of the glycopeptide to the native receptor is minimally affected. Therefore, the glycosylated Ang-(1-7) oligopeptide derivatives, at a minimum, maintain Mas binding similar to that of the native Ang-(1-7) peptide. In addition, promoting the aqueous nature of the glycopeptide can further enhance vascular efficacy of Ang-(1-7) oligopeptide derivatives. The degree of glycosylation (e.g., Table 2. Unglycosylated Ser^{o}, glucosylated Ser*, or lactosylated Ser**) for optimal blood-brain barrier transport is determined using the best binding compounds from these using the in vivo mouse model. Besides the disaccharide β-lactose, the more robust disaccharide β-cellobiose is examined using these first few structures. Based on the amino acid sequence of Ang-(1-7) and the potential modification strategies, there are at least about 200 possible derivatives of the Ang-(1-7) oligopeptide that are rapidly generated using the well-known oligopeptide synthesis, including automated peptide synthesis as well as combinatorial synthesis.

### Amyloid-Related Imaging Abnormalities

AD is a neurodegenerative disorder that is believed to be, in part, caused by misfolding of beta-amyloid (Aβ) monomers and the resulting accumulation of toxic, soluble Aβ oligomers. Accordingly, several monoclonal antibodies against Aß have been developed for treatment of AD (“anti-Aβ antibodies”). The first of this class of antibodies to enter clinical trials was bapineuzumab, whose binding to the N-terminus of Aβ monomers and aggregates activates microglial-mediated phagocytosis. Solanezumab binds the mid-domain of AB (residues 16-26) and gantenerumab binds the N-terminus and central region of Aβ (residues 18-27), both of which were designed to remove AB monomers. Crenezumab binds AB monomers and aggregates and was designed to reduce the release of proinflammatory cytokines. Other antibodies that belong to this class include ponezumab (binds C-terminus of Aβ), donanemab (binds Aβ), lecanemab (binds Aβ), and most recently, aducanumab (binds N-terminus of Aβ). Lecanemab and aducanumab are currently being pursued further as a treatment for AD. While these amyloid-modifying monoclonal antibodies have different epitopes and mechanisms of action, all have been shown to lead to the development of amyloid-related imaging abnormalities (ARIA) in AD patients.

ARIA refers to a spectrum of signal changes that are detected in MRI sequences of AD patients. There are two types of ARIA. ARIA-effusion (ARIA-E) is characterized by the presence of vasogenic edema (VE) and effusion. ARIA-hemorrhages (ARIA-H) is characterized by the presence of microhemorrhages (mH) and is often presented with hemosiderosis. ARIA may be observed in AD patients. In some embodiments, ARIA is observed in AD patients who are receiving antibody therapies. Of the monoclonal antibodies mentioned above, it has been shown that bapineuzumab often leads to the development of ARIA-E and that other antibodies often lead to the development of both ARIA-E and ARIA-H in the subject.

The exact mechanism by which antibody therapies lead to the development of ARIA in AD patients has not been elucidated. However, for amyloid-modifying monoclonal antibody therapies, it has been postulated that the amyloid-modifying monoclonal antibodies affect the ability of amyloid to maintain the integrity of the vessel wall and/or the blood-brain barrier, or that the amyloid-modifying antibodies trigger an inflammatory response which, in turn, results in the development of ARIA-E and ARIA-H.

When ARIA does present with symptoms, they are non-specific and include headache, confusion, visual disturbances, visuospatial impairment, praxis difficulties, and neuropsychiatric symptoms. ARIA and associated symptoms are resolved when patients discontinue the antibody treatment.

### Methods to Mitigating Effects of ARIA

The invention described herein provides methods of mitigating the effects of ARIA in a subject who has been receiving an antibody therapy for AD by administering an Ang-(1-7) oligopeptide derivative to the subject.

The Ang-(1-7) oligopeptide derivative may be administered before the subject is first administered an antibody therapy. In some embodiments, the subject may be administered the Ang-(1-7) oligopeptide derivative for a period of days or weeks prior to being administered an antibody therapy. For example, the Ang-(1-7) oligopeptide derivative may be administered to the subject for a period of 1 day to 6 months (e.g., 1 day and 5 months, 1 day and 4 months, 1 day and 3 months, 1 day and 2 months, 1 day and 1 month, 1 day and 2 weeks, 1 day and 1 week, 1 week and 6 months, 1 month and 6 months, 2 months and 6 months, 3 months and 6 months, 4 months and 6 months, 5 months and 6 months, 2 weeks and 2 months, or 1 week and 1 month) before they are receive an administration of the antibody therapy for the treatment of AD. In some embodiments, the Ang-(1-7) oligopeptide derivative is administered to the subject from 1 week to 3 months (e.g., 1 week and 10 weeks, 1 week and 8 weeks, 1 week and 6 weeks, 1 week and 4 weeks, 1 week and 2 weeks, 2 weeks and 3 months, 4 weeks and 3 months, 6 weeks and 3 months, 8 weeks and 3 months, 10 weeks and 3 months, or 3 weeks and 6 weeks) prior to when the antibody therapy for the treatment of Alzheimer's disease is first administered to the subject. The Ang-(1-7) oligopeptide derivative may be administered to the subject from 1 week to 2 months (e.g., 1 week and 6 weeks, 1 week and 5 weeks, 1 week and 4 weeks, 1 week and 3 weeks, 1 week and 2 weeks, 2 weeks and 8 weeks, 3 weeks and 8 weeks, 4 weeks and 8 weeks, 5 weeks and 8 weeks, 6 weeks and 8 weeks, or 7 weeks and 8 weeks) prior to when the antibody therapy for the treatment of Alzheimer's disease is first administered to the subject.

The Ang-(1-7) oligopeptide derivative may be administered to the subject for any period of determined to be suitable by a clinician. In some embodiments, the Ang-(1-7) oligopeptide derivative is administered to the subject for at least 1 month. In some embodiments, the Ang-(1-7) oligopeptide derivative is administered to the subject for a period of from about 1 month to about 2 years (e.g., 1 month and 18 months, 1 month and 12 months, 1 month and 10 months, 1 month and 6 months, 1 month and 4 months, 2 months and 2 year, 6 months and 2 years, 12 months and 2 years, 18 months and 2 years, or 6 months and 12 months). For example, the Ang-(1-7) oligopeptide derivative may be administered to the subject for a period of from about 1 month to about 1 year (e.g., 1 month and 10 months, 1 month and 8 months, 1 month and 6 months, 1 month and 4 months, 1 month and 2 months, 2 months and 1 year, 4 months and 1 year, 6 months and 1 year, 8 months and 1 year, 10 months and 1 year, or 4 months and 10 months). In some embodiments, the Ang-(1-7) oligopeptide derivative is administered to the subject for the entire period the subject is also receiving the antibody therapy for the treatment of AD. In some embodiments, the Ang-(1-7) oligopeptide derivative and the antibody therapy for the treatment of AD are administered concurrently.

### Formulations

The Ang-(1-7) oligopeptide derivatives described herein may be administered to the subject in the form of a pharmaceutical composition comprising at least one Ang-(1-7) receptor agonist or a pharmaceutically acceptable salt or solvate thereof, together with at least one pharmaceutically acceptable carrier, and optionally other therapeutic and/or prophylactic ingredients.

Typically, the Ang-(1-7) oligopeptide derivatives are administered as pharmaceutical formulations including those suitable for oral (including buccal and sub-lingual), nasal, pulmonary, or parenteral (including intramuscular, intraarterial, intrathecal, subcutaneous, and intravenous) administration or in a form suitable for administration by inhalation or insufflation. Typical manner of administration is generally oral or parenteral using a convenient dosage regimen which can be adjusted according to the degree of affliction. Compositions of the invention may include one or more conventional adjuvants, carriers, or diluents, and can be placed into the form of pharmaceutical compositions and unit dosages. The pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of sterile injectable solutions for parenteral use. Formulations containing about 100 milligrams of active ingredient or, more broadly, about 1 to about 1,000 milligrams, per tablet, are accordingly suitable representative unit dosage forms.

The Ang-(1-7) oligopeptide derivatives may be orally administered, for example, with an inert diluent or with an assimilable edible carrier, or it can be enclosed in hard or soft shell gelatin capsules, or it can be compressed into tablets. For oral therapeutic administration, the active oligopeptide may be incorporated with excipient and used in the form of ingestible tablets, buccal tablets, troches, capsules, elixirs, suspensions, syrups, wafers, and the like. Such compositions and preparation can contain at least 0.1% of the active oligopeptide. The percentage of the compositions and preparation can, of course, be varied and can conveniently be between about 1 to about 10% of the weight of the unit. The amount of active oligopeptide in such therapeutically useful compositions is such that a suitable dosage will be obtained. Preferred compositions or preparations according to the present invention are prepared such that an oral dosage unit form contains from about 1 to about 1000 mg of active oligopeptide.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active oligopeptide, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active oligopeptide can be incorporated into sustained-release preparations and formulation.

The active oligopeptide can also be administered parenterally. Solutions of the active oligopeptide can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacteria and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the active oligopeptide in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The Ang-(1-7) oligopeptide derivatives described herein can be administered to a subject alone or in combination with pharmaceutically acceptable carriers, as noted above, the proportion of which is determined by the solubility and chemical nature of the oligopeptide, chosen route of administration and standard pharmaceutical practice.

### Extended-Release Formulations

The Ang-(1-7) oligopeptide derivative which is administered to the subject to mitigate the effects of ARIA may be formulated as an extended-release injectable gel. The extended-release injectable gel may be formulated according to International Publication number WO 2022/216941, which is incorporated herein by reference in its entirety.

The extended-release injectable gel may include a biocompatible polymer (e.g., homopolymer or co-polymer). In some embodiments, polymers suitable for use in conjunction with the disclosed compositions include linear polyesters. The linear polyesters may be prepared from a-hydroxy carboxylic acids, e.g., lactic acid and/or glycolic acid, by condensation of the lactone dimers, see e.g. U.S. Pat. No. 3,773,919, the contents of which are incorporated herein by reference. The polyester chains in the linear polymers may be copolymers of the a-carboxylic acid moieties, lactic acid and glycolic acid, or of the lactone dimers.

In some embodiments, the biocompatible polymer may be sucrose acetoisobutyrate (SAIB). SAIB is an FDA-approved food additive that exhibits safe human daily intake of up to 20 mg/kg. Once in the body, SAIB is metabolized into sucrose and partially acylated sucrose, which are readily cleared from the body. SAIB is a highly hydrophobic, viscous liquid that forms a low-viscosity solution when mixed in certain organic solvents.

Linear polyesters, e.g., linear PLGA, that may be used according to the present disclosure have a weight average molecular weight (MW) between about 10,000 and about 500,000 Da, e.g.

between about 47,000 to about 63,000, between about 24,000 and 38,000, between about 10,000 and 8,000, between about 4,000 and 15,000, or between about 7,000 and 17,000. Such polymers have a polydispersity M_{w}/Mₙ e.g. between 1.2 and 2. Suitable examples include e.g. poly (D,L-lactide-co-glycolide), e.g. having a general formula —[(C₆H₈O₄)ₓ(C₄H₄O₄)_{y}]ₙ— (each of x, y and n having a value so that the total sum gives the above indicated MWs), e.g. those commercially available, e.g. Resomers® from Boehringer Ingelheim, in particular Resomers® RG, e.g. Resomer® RG 502, 502H, 503, 503H, 504, 504H.

In some embodiments, the biocompatible polymers suitable for use with the formulations disclosed herein (e.g., PLA or PLGA) may be end-capped with esters or with a free carboxylic acid. Generally, ester-capped polymers exhibit a longer degradation half-life as compared to acid-capped polymers. In some embodiments, the biocompatible polymer of the disclosure is ester-capped. In some embodiments, the biocompatible polymer of the disclosure is acid-capped (e.g., carboxylic acid-capped).

The molar ratio of lactide: glycolide of PLGA in the formulation of the disclosure may be from about 100:0 to 25:75 (e.g., 100:0, 90:10, 80:20, 75:25, 60:40, 50:50, 40:60, 30:70, or 25:75). In some embodiments, the molar ratio of lactide: glycolide in a PLGA co-polymer is 100:0 (e.g., PLA), 75:25, or 50:50. In some embodiments, the molar ratio of lactide: glycolide in a PLGA co-polymer is 100:0. In some embodiments, the molar ratio of lactide: glycolide in a PLGA co-polymer is 75:25. In some embodiments, the molar ratio of lactide: glycolide in a PLGA co-polymer is 50:50.

In some embodiments, the extended-release gel formulations include a specific ratio of the therapeutic Ang-(1-7) oligopeptide derivatives to the biocompatible polymer (e.g., PLA or PLGA). In some embodiments, the gel formulation contains an Ang-(1-7) oligopeptide derivative and a biocompatible polymer at a ratio of 3:1. In some embodiments, the gel formulation contains an Ang-(1-7) oligopeptide derivative and a biocompatible polymer at a ratio of 3:2. In some embodiments, the gel formulation contains an Ang-(1-7) oligopeptide derivative and a biocompatible polymer at a ratio of 1:1. In some embodiments, the gel formulation contains an Ang-(1-7) oligopeptide derivative and a biocompatible polymer at a ratio of 2:3. In some embodiments, the gel formulation contains an Ang-(1-7) oligopeptide derivative and a biocompatible polymer at a ratio of 1:3. In some embodiments, the gel formulation contains an Ang-(1-7) oligopeptide derivative and a biocompatible polymer at a ratio of 1:2. Other ratios of the Ang-(1-7) oligopeptide derivatives and the biocompatible polymer not described herein may be used.

In some embodiments, the extended-release gel formulations of the disclosure may be prepared by dissolving the biocompatible polymer (e.g., PLA or PLGA) and the biologically-active therapeutic agent (i.e., Ang-(1-7) oligopeptide derivatives) in a solvent, such as, e.g., an organic solvent or aqueous solvent. The biocompatible polymer and the biologically-active therapeutic agent may be separately dissolved using the same type of solvent and then combined together. Alternatively, the biocompatible polymer and the biologically-active therapeutic agent may be dissolved together in the same admixture containing the solvent.

As the organic solvent, dimethyl sulfoxide (DMSO), oleic acid, ethoxylated castor oil, ethyl oleate, triacetin, ethyl laureate, triethyl citrate, polyethylene glycol 300, N-methyl-2-pyrrolidone (NMP), benzoic acid (BzOH), benzyl alcohol (BA), benzyl benzoate (BB), dimethylacetamide (DMA), dichloromethane, chloroform, dichloroethane, trichloroethane, carbon tetrachloride, ethyl ether, isopropyl ether, ethyl acetate, butyl acetate, benzene, toluene, xylene, ethanol, methanol, acetonitrile and the like may be used. These may be used in admixture of appropriate ratios. For example, different ratios of the above solvents may be combined to achieve optimal solubility of the biocompatible polymer and the biologically-active therapeutic agent. In a non-limiting example, DMSO and BzOH may be combined in a 1:1 (v/v) ratio to dissolve the polymer and the therapeutic agent. In another example, DMSO and BB are combined at a 75%: 25% (v/v) ratio. Furthermore, certain solvents made be added as excipients. In particular, oleic acid, palmitic acid, and myristic acid may be added as excipients. Water-insoluble oils, such as, e.g., benzyl benzoate, ethoxylated castor oil, palm oil, ethyl oleate, triacetin, ethyl laureate, triethyl citrate, polyethylene glycol (PEG) 300, dimethylacetamide (DMA) may be added as excipients to primary solvents (e.g., DMSO, NMP, BB, etc.).

Relatedly, various aqueous buffers may also be used as a vehicle for testing the in vitro release properties of the disclosed formulations. Once the formulation is added to these aqueous buffers, it generally forms a gel depot from which the active ingredient (e.g., an oligopeptide disclosed herein) is released. Non-limiting examples of suitable aqueous vehicles include PBS, sterile saline solution (e.g., 0.9% NaCl solution), sterile water, lactated Ringer solution, Ringer's acetate, sodium-bicarbonate solution, among others.

In a particular example, the extended-release gel formulation containing a biocompatible polymer (e.g., PLA or PLGA) having dispersed therein an effective amount of the oligopeptide, such that the formulation exhibits a sustained release in vitro of at least 60% of the effective amount of the therapeutic agent within 48 hours following placement of the formulation in a release medium (e.g., PBS at 37° C., pH 7.4) at a time=0 (t₀) and an initial burst release not greater than 30% of the effective amount of the oligopeptide within 24 hours following t₀. The formulation may be configured to have a sustained release profile that does not exceed an average rate of release of the oligopeptide that is greater than 20%/24 hours for a period that is equal to or greater than seven days following to, as measured by high performance liquid chromatography (HPLC) and/or mass spectrometry (MS) at an operating temperature of 37° C.

In another example, the extended-release gel formulation containing a biocompatible polymer and an effective amount of an oligopeptide, such that the formulation exhibits a sustained release in vitro of at least 60% of the effective amount of the therapeutic agent within 25 days following placement of the formulation in a release medium (e.g., PBS at 37° C., pH 7.4) at a time=0 (t₀) and an initial burst release not greater than 30% of the effective amount of the oligopeptide within 24 hours following to. The formulation may be configured to have a sustained release profile that does not exceed an average rate of release of the oligopeptide that is greater than 30%/24 hours for a period that is equal to or greater than seven days following to, as measured by HPLC and/or MS at an operating temperature of 37° C.

In the context of in vivo administration to a subject (e.g., a human), the extended-release gel formulation contains a biocompatible polymer and an effective amount of a biologically-active therapeutic agent, such that, following subcutaneous or intramuscular administration to a human subject, the formulation is configured to form a depot in vivo that releases the oligopeptide at a rate sufficient to maintain an average serum concentration that is between 1-1000 ng/ml for a period of 24-168 hours following administration, a maximum serum concentration (Cₘₐₓ) of the oligopeptide that is between 1-1000 ng/ml for a period of 48 hours following administration.

In some embodiments, the extended-release gel formulation contains a biocompatible polymer having dispersed therein an effective amount of an oligopeptide, such that, following subcutaneous or intramuscular administration to a human subject, the formulation is configured to form a depot in vivo that releases the oligopeptide at a rate sufficient to maintain an average serum concentration of between 1 and 1000 ng/ml for a period of 21 days following administration, a Cₘₐₓ of the oligopeptide of between 1 and 1000 ng/ml for a period of 21 days following administration. Routine and well-known methods may be employed to determine in vivo pharmacokinetic parameters in human subjects.

### Dosing

A clinician may determine the dosage of the Ang-(1-7) oligopeptide derivatives which will be most suitable for mitigating the effects of ARIA in a subject and it will vary with the form of administration and the particular oligopeptide chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

In some embodiments, the Ang-(1-7) oligopeptide derivatives described herein are administered in a therapeutically effective amount by any of the accepted modes of administration for agents that serve similar utilities. Suitable dosage ranges are typically 1-500 mg daily, typically 1-100 mg daily, and often 1-30 mg daily, depending on numerous factors, e.g., the severity of the cognitive impairment, the age and relative health of the subject, the potency of the Ang-(1-7) receptor agonist used, the route and form of administration, the indication towards which the administration is directed, and the preferences and experience of the medical practitioner involved. In some embodiments, the Ang-(1-7) oligopeptide derivative may be administered to the subject one a day, twice a day, three times a day, or four times a day. One of ordinary skill in the art of treating such diseases is typically able, without undue experimentation and in reliance upon personal knowledge and the disclosure of this application, to ascertain a therapeutically effective amount of oligopeptide.

### Extended-Release Dosing Regimen

The disclosed formulation or a pharmaceutically acceptable suspension thereof may be administered to a subject (e.g., a human) such that a therapeutically effective amount of an Ang-(1-7) oligopeptide derivative is delivered to the subject, such as, e.g., from about 0.1 to about 1000 mg/day (e.g., 10-100 mg/day, 0.1-50 mg/kg of body weight/day). In some embodiments, the extended-release injectable gel formulation is administered to the subject at least once a year. In some embodiments, the extended-release injectable gel formulation is administered to the subject from once a week to once a year (e.g., once a week to every 2 months, once a week to every 4 months, once a week to every 6 months, once a week to every 8 months, once a week to every 10 months, once a month to once a year, once every 2 months to once a year, once every 4 months to once a year, once every 6 months to once a year, once every 8 months to once a year, or once a month to once every two months). For example, the extended-release injectable gel formulation may be administered to the subject from once a 1 week to once a month (e.g., once a week, every 2 weeks, every 3 weeks, every 4 weeks).

### Kits

The disclosure also provides kits that include either a daily administration formulation or an extended-release gel formulation, or a pharmaceutical composition containing the same for use in the prevention or treatment of ARIA. The kits can optionally include an agent or device for delivering the agent to the subject. In other examples, the kits may include one or more sterile applicators, such as syringes or needles. Further, the kits may optionally include other agents, e.g., anesthetics or antibiotics. The kit can also include a package insert that instructs a user of the kit, such as a physician, to perform the methods disclosed herein.

### EXAMPLES

### Example 1

### Daily Administration of an Ang-(1-7) Oligopeptide Derivative to Mitigate the Effects of ARIA in a Subject Receiving an Antibody Therapy for the Treatment of Alzheimer's Disease

Subjects receiving an antibody therapy for the treatment of AD may be administered a therapeutically effective amount of an Angiotensin-(1-7) (Ang-(1-7)) receptor agonist to mitigate the effects of amyloid-related imaging abnormalities (ARIA) in the subject. In this example, the Ang-(1-7) receptor agonist is administered daily to mitigate the effects of ARIA in the subject.

According to the methods disclosed herein, the oligopeptide that is administered to the subject is an Ang-(1-7) oligopeptide derivative of the formula:

(SEQ ID NO: 1)
A¹-A²-A³-A⁴-A⁵-A⁶-A⁷-A⁸,

where A¹ is selected from the group consisting of aspartic acid, glutamic acid, alanine, and glycosylated forms thereof; A² is selected from the group consisting of arginine, histidine, lysine, and glycosylated forms thereof; A³ is selected from the group consisting of valine, alanine, isoleucine, leucine, and glycosylated forms thereof; A⁴ is selected from the group consisting of tyrosine, phenylalanine, tryptophan, and glycosylated forms thereof; A⁵ is selected from the group consisting of isoleucine, valine, alanine, leucine, and glycosylated forms thereof; A⁶ is selected from the group consisting of histidine, arginine, lysine, and glycosylated forms thereof; A⁷ is selected from the group consisting of proline, glycine, serine, and glycosylated forms thereof; and A⁸ can be present or absent, wherein when A⁸ is present, A⁸ is selected from the group consisting of serine, threonine, hydroxyproline, and glycosylated forms thereof, provided (i) at least one of A¹-A⁸ is optionally substituted with a mono-or di-carbohydrate; or (ii) when A⁸ is absent: (a) at least one of A¹-A⁷ is substituted with a mono-or di-carbohydrate, (b) A⁷ is terminated with an amino group, or (c) a combination thereof. A¹ may be the amino terminal end of the oligopeptide and A⁸ (or A⁷ when A⁸ is absent) may be the carboxyl terminal end. A¹ may be the carboxyl terminal end and A⁸ (or A⁷ when

A⁸ is absent) may be the amino terminal end. The Ang-(1-7) oligopeptide derivative administered to the subject may have the amino acid sequence of any one of SEQ ID NOs: 6-13.

The Ang-(1-7) oligopeptide derivative may be administered to a subject who is receiving an antibody therapy for the treatment of AD. The antibody therapy may be a monoclonal antibody therapy. For example, the antibody therapy may include administration of an anti-beta-amyloid antibody to the subject, wherein the antibody may be bapineuzumab, solanezumab, gantenerumab, crenezumab, ponezumab, lecanemab, donanemab, and aducanumab, or a combination thereof.

The Ang-(1-7) oligopeptide derivative may be administered to the subject prior to the subject receiving a first administration of the antibody therapy. The Ang-(1-7) oligopeptide derivative may be administered one day prior to the subject being administered an antibody therapy. The Ang-(1-7) oligopeptide derivative may be administered to the subject for a period of 1 day to 6 months before they receive an administration of the antibody therapy for the treatment of AD. The Ang-(1-7) oligopeptide derivative may be administered to the subject from 1 week to 3 months prior to when the antibody therapy for the treatment of AD is first administered to the subject. The Ang-(1-7) oligopeptide derivative may be administered to the subject from 1 month prior to when the antibody therapy for the treatment of Alzheimer's disease is first administered to the subject.

The Ang-(1-7) oligopeptide derivative may be administered to the subject for at least 1 month. The Ang-(1-7) oligopeptide derivative may be administered to the subject for a period of from about 1 month to about 2 years. For example, the Ang-(1-7) oligopeptide derivative may be administered to the subject for a period of from about 1 month to about 1 year. The Ang-(1-7) oligopeptide derivative may be administered to the subject for the entire period the subject is also receiving the antibody therapy for the treatment of AD.

The Ang-(1-7) oligopeptide derivative may be administered to the subject in an amount of about 0.1 to about 1000 mg/day, and preferably from about 10 to about 100 mg/day, or from about 0.1 to about 50 mg/Kg of body weight per day and preferably from about 0.1 to about 20 mg/Kg of body weight per day. The Ang-(1-7) oligopeptide derivative may be administered to the subject once a day, twice a day, three times a day, or four times a day. The Ang-(1-7) oligopeptide derivative may be administered to the subject intravenously, subcutaneously, intramuscularly, intraperitoneally, intracerebroventricularly, or by intrathecal injection.

### Example 2

### Daily Administration of an Ang-(1-7) Oligopeptide Derivative to Mitigate the Effects of ARIA in a Subject Receiving an Antibody Therapy for the Treatment of Alzheimer's Disease

Subjects receiving an antibody therapy for the treatment of AD may be administered a therapeutically effective amount of an Ang-(1-7) receptor agonist to mitigate the effects of amyloid-related imaging abnormalities (ARIA) in the subject. In this example, the Ang-(1-7) receptor agonist is administered using an extended-release gel formulation to mitigate the effects of ARIA in the subject, while administering the Ang-(1-7) receptor agonist once a week or less.

According to the methods disclosed herein, the oligopeptide that is administered to the subject is an Ang-(1-7) oligopeptide derivative of the formula:

(SEQ ID NO: 1)
A¹-A²-A³-A⁴-A⁵-A⁶-A⁷-A⁸,

where A¹ is selected from the group consisting of aspartic acid, glutamic acid, alanine, and glycosylated forms thereof; A² is selected from the group consisting of arginine, histidine, lysine, and glycosylated forms thereof; A³ is selected from the group consisting of valine, alanine, isoleucine, leucine, and glycosylated forms thereof; A⁴ is selected from the group consisting of tyrosine, phenylalanine, tryptophan, and glycosylated forms thereof; A⁵ is selected from the group consisting of isoleucine, valine, alanine, leucine, and glycosylated forms thereof; A⁶ is selected from the group consisting of histidine, arginine, lysine, and glycosylated forms thereof; A⁷ is selected from the group consisting of proline, glycine, serine, and glycosylated forms thereof; and A⁸ can be present or absent, wherein when A⁸ is present, A⁸ is selected from the group consisting of serine, threonine, hydroxyproline, and glycosylated forms thereof, provided (i) at least one of A¹-A⁸ is optionally substituted with a mono-or di-carbohydrate; or (ii) when A⁸ is absent: (a) at least one of A¹-A⁷ is substituted with a mono-or di-carbohydrate, (b) A⁷ is terminated with an amino group, or (c) a combination thereof. A¹ may be the amino terminal end of the oligopeptide and A⁸ (or A⁷ when A⁸ is absent) may be the carboxyl terminal end. A¹ may be the carboxyl terminal end and A⁸ (or A⁷ when A⁸ is absent) may be the amino terminal end. The Ang-(1-7) oligopeptide derivative administered to the subject may have the amino acid sequence of any one of SEQ ID NOs: 6-13.

The Ang-(1-7) oligopeptide derivative may be administered to a subject who is receiving an antibody therapy for the treatment of AD. The antibody therapy may be a monoclonal antibody therapy. For example, the antibody therapy may include administration of an anti-beta-amyloid antibody to the subject, wherein the antibody may be bapineuzumab, solanezumab, gantenerumab, crenezumab, ponezumab, lecanemab, donanemab, and aducanumab, or a combination thereof.

The Ang-(1-7) oligopeptide derivative may be administered to the subject prior to the subject receiving a first administration of the antibody therapy. The Ang-(1-7) oligopeptide derivative may be administered one day prior to the subject being administered an antibody therapy. The Ang-(1-7) oligopeptide derivative may be administered to the subject for a period of 1 day to 6 months before they receive an administration of the antibody therapy for the treatment of AD. The Ang-(1-7) oligopeptide derivative may be administered to the subject from 1 week to 3 months prior to when the antibody therapy for the treatment of AD is first administered to the subject. The Ang-(1-7) oligopeptide derivative may be administered to the subject from 1 month prior to when the antibody therapy for the treatment of Alzheimer's disease is first administered to the subject.

The Ang-(1-7) oligopeptide derivative may be administered to the subject for at least 1 month. The Ang-(1-7) oligopeptide derivative may be administered to the subject for a period of from about 1 month to about 2 years. For example, the Ang-(1-7) oligopeptide derivative may be administered to the subject for a period of from about 1 month to about 1 year. The Ang-(1-7) oligopeptide derivative may be administered to the subject for the entire period the subject is also receiving the antibody therapy for the treatment of AD.

The Ang-(1-7) oligopeptide derivative may be formulated as an extended-release gel, wherein the Ang-(1-7) oligopeptide derivative is formulated with a biopolymer as described herein. The extended-release formulation may include a poly-lactic acid (PLA), poly-(lactic-co-glycolic acid) (PLGA) polymer matrix, or sucrose acetoisobutyrate (SAIB) polymer having dispersed therein an effective amount (e.g., a therapeutically effective amount) of the Ang-(1-7) oligopeptide derivatives. The formulation may also contain a solvent (e.g., an organic solvent) for dissolving the biocompatible polymer and the Ang-(1-7) oligopeptide derivatives.

The extended-release formulation may be administered to a subject (e.g., a human) such that an amount of about 0.1 to about 1000 mg/day (e.g., 10-100 mg/day, 0.1-50 mg/kg of body weight/day). The extended-release injectable gel formulation may be administered to the subject at least once a year. The extended-release injectable gel formulation is administered to the subject from once a week to once a year. For example, the extended-release injectable gel formulation may be administered to the subject from once a 1 week to once a month. The Ang-(1-7) oligopeptide derivative may be administered to the subject intravenously, subcutaneously, intramuscularly, intraperitoneally, intracerebroventricularly, or by intrathecal injection.

### Example 3

### Effect of Administration of PNA5 on ARIA

The effect the Ang-(1-7) oligopeptide PNA5 had on cerebral microbleeds (ARIA-H) in a mouse model of AD and cerebral amyloid angiopathy (CAA) was studied.

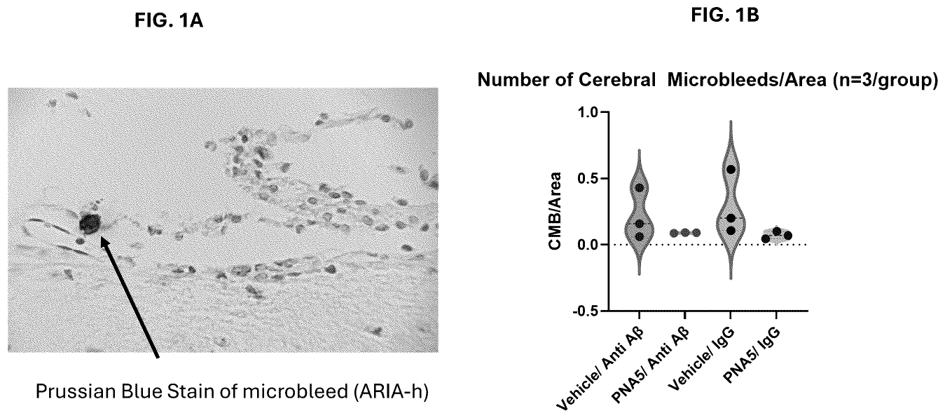

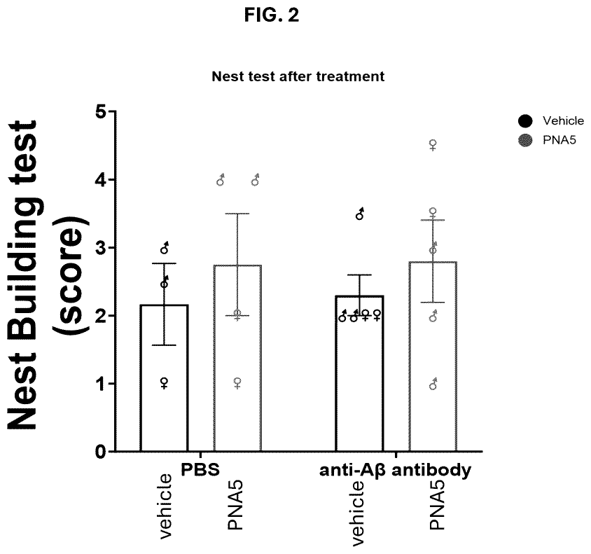

Tg-SwDI mice aged 12-13 months were administered: (1) the PNA5 vehicle, without PNA5, and PBS, (2) vehicle and an anti-Aβ antibody, (3) PNA5 and PBS, or (4) PNA5 and an anti-Aβ antibody. The anti-AB antibody therapy (IgG) was administered in an amount of 3 mg/kg. The PNA5 was administered in an amount of 30 μL. For groups receiving the PNA5 alone (Group 3), the compounds were administered for 2 at which point they were administered a Norwegian Tenecteplase Stroke Trial (NOR) test and Nest building test (FIG. 2) prior to euthanasia and collection of the tissue for analysis of the microbleeds (FIGS. 1A and 1B). For groups receiving the anti-AB antibody and PNA5 (Group 4), the compounds were administered for 2 months at which point they were administered a Norwegian Tenecteplase Stroke Trial (NOR) test and Nest building test prior (FIG. 2) to euthanasia and collection of the tissue for analysis of the microbleeds (FIGS. 1A and 1B). The resulting data demonstrated that the administration of PNA5 decreased cerebral microbleeds (ARIA-H) in the Tg-SwDI mouse model of AD and cerebral amyloid angiopathy (CAA).

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A method for mitigating the effects of amyloid-related imaging abnormalities (ARIA) in a subject receiving an antibody therapy for the treatment of Alzheimer's disease, wherein the method comprises administering a therapeutically effective amount of an Angiotensin-(1-7) (Ang-(1-7)) receptor agonist to the subject.

2. The method of claim 1, wherein the antibody therapy comprises administration of one or more antibodies to the subject for the treatment of Alzheimer's disease.

3. The method of claim 2, wherein the one or more antibodies comprises a monoclonal antibody.

4. The method of claim 2, wherein the one or more antibodies comprises an anti-beta-amyloid antibody.

5. The method of claim 2, wherein the one or more antibodies is selected from bapineuzumab, solanezumab, gantenerumab, crenezumab, ponezumab, lecanemab, donanemab, and aducanumab, or a combination thereof.

6. The method of any one of claims 1-5, wherein the Ang-(1-7) receptor agonist comprises an Ang-(1-7) oligopeptide derivative.

7. The method of claim 6, wherein the Ang-(1-7) oligopeptide derivative has the formula: A¹-A²-A³-A⁴-A⁵-A⁶-A⁷-A⁸ (SEQ ID NO: 1) wherein:
A¹ is selected from the group consisting of aspartic acid, glutamic acid, alanine, and glycosylated forms thereof;
A² is selected from the group consisting of arginine, histidine, lysine, and glycosylated forms thereof;
A³ is selected from the group consisting of valine, alanine, isoleucine, leucine, and glycosylated forms thereof;
A⁴ is selected from the group consisting of tyrosine, phenylalanine, tryptophan, and glycosylated forms thereof;
A⁵ is selected from the group consisting of isoleucine, valine, alanine, leucine, and glycosylated forms thereof;
A⁶ is selected from the group consisting of histidine, arginine, lysine, and glycosylated forms thereof;
A⁷ is selected from the group consisting of proline, glycine, serine, and glycosylated forms thereof; and
A⁸ can be present or absent, wherein when A⁸ is present, A⁸ is selected from the group consisting of serine, threonine, hydroxyproline, and glycosylated forms thereof.

8. The method of claim 7, wherein at least one of A¹-A⁸ is glycosylated with a monosaccharide or disaccharide.

9. The method of claim 7, wherein A⁸ is glycosylated with a monosaccharide or disaccharide or A⁸ is absent and A⁷ is glycosylated with a monosaccharide or disaccharide.

10. The method of any one of claims 7-9, wherein at least one of the monosacharides or disaccharides is selected from the group consisting of glucose, galactose, xylose, fucose, rhamnose, lactose, cellobiose, and melibiose.

11. The method of any one of claims 7-10, wherein (a) A⁷ is a serine or a glycosylated form thereof and A⁸ is absent or (b) A⁸ is serine or a glycosylated form thereof.

12. The method of any one of claims 7-11, wherein (a) A⁷ is glycosylated with glucose or lactose and A⁸ is absent or (b) A⁸ is glycosylated with glucose or lactose.

13. The method of any one of claims 7-12, wherein (a) A⁷ is terminated with an amino group and A⁸ is absent or (b) A⁸ is terminated with an amino group.

14. The method of claim 6, wherein the Ang-(1-7) oligopeptide derivative is selected from the group consisting of any one of SEQ ID NOs: 6-13.

15. The method of claim 14, wherein the Ang-(1-7) oligopeptide derivative is SEQ ID NO: 6.

16. The method of claim 14, wherein the Ang-(1-7) oligopeptide derivative is SEQ ID NO: 7.

17. The method of claim 14, wherein the Ang-(1-7) oligopeptide derivative is SEQ ID NO: 8.

18. The method of claim 14, wherein the Ang-(1-7) oligopeptide derivative is SEQ ID NO: 9.

19. The method of claim 14, wherein the Ang-(1-7) oligopeptide derivative is SEQ ID NO: 10.

20. The method of claim 14, wherein the Ang-(1-7) oligopeptide derivative is SEQ ID NO: 11.

21. The method of claim 14, wherein the Ang-(1-7) oligopeptide derivative is SEQ ID NO: 12.

22. The method of claim 14, wherein the Ang-(1-7) oligopeptide derivative is SEQ ID NO: 13.

23. The method of any one of claims 6-22, wherein the Ang-(1-7) oligopeptide derivative comprises at least one D-amino acid.

24. The method of claim 23, wherein each amino acid is a D-amino acid.

25. The method of any one of claims 1-24, wherein the Ang-(1-7) oligopeptide derivative is formulated as an injectable solution or dispersion.

26. The method of any one of claims 1-25, wherein the Ang-(1-7) oligopeptide derivative is administered to the subject intravenously, subcutaneously, intramuscularly, intraperitoneally, intracerebroventricularly, or by intrathecal injection.

27. The method of any one of claims 1-26, wherein the Ang-(1-7) oligopeptide derivative is administered to the subject in an amount of 0.1 mg/day to 1000 mg/day.

28. The method of claim 27, wherein the Ang-(1-7) oligopeptide derivative is administered to the subject in an amount of 0.1-50 mg/kg of the subject's body weight per day.

29. The method of any one of claims 1-28, wherein the Ang-(1-7) oligopeptide derivative is administered to the subject 1 to 4 times daily.

30. The method of claim 24, wherein the Ang-(1-7) oligopeptide derivative is administered to the subject once a day.

31. The method of any one of claims 1-30, wherein the Ang-(1-7) oligopeptide derivative is formulated as an extended-release injectable gel.

32. The method of claim 31, wherein the extended-release injectable gel formulation is administered to the subject intravenously, subcutaneously, intramuscularly, intraperitoneally, intracerebroventricularly, or by intrathecal injection.

33. The method of claim 31 or claim 32, wherein the extended-release injectable gel formulation is administered to the subject at least once a year.

34. The method of claim 33, wherein the extended-release injectable gel formulation is administered to the subject from once a 1 week to once a year.

35. The method of claim 34, wherein the extended-release injectable gel formulation is administered to the subject from once a 1 week to once a month.

36. The method of any one of claims 31-35, wherein the Ang-(1-7) oligopeptide derivative is administered to the subject in amount of 10-14 mg/day, 70 mg/week, 140 mg/two weeks, or 280 mg/month.

37. The method of any one of claims 31-36, wherein the extended-release injectable gel formulation is administered to the subject through a needle having a diameter of 20 to 25 gauge.

38. The method of any one of claims 1-37, wherein the Ang-(1-7) oligopeptide derivative is administered to the subject from 1 day to 6 months prior to when the antibody therapy for the treatment of Alzheimer's disease is first administered to the subject.

39. The method of claim 38, wherein the Ang-(1-7) oligopeptide derivative is administered to the subject from 1 week to 3 months prior to when the antibody therapy for the treatment of Alzheimer's disease is first administered to the subject.

40. The method of claim 39, wherein the Ang-(1-7) oligopeptide derivative is administered to the subject from 1 week to 2 months prior to when the antibody therapy for the treatment of Alzheimer's disease is first administered to the subject.

41. The method of any one of claims 1-40, wherein the Ang-(1-7) oligopeptide derivative is administered to the subject for at least 1 month.

42. The method of claim 41, wherein the Ang-(1-7) oligopeptide derivative is administered to the subject for a period of from about 1 month to about 2 years.

43. The method of claim 42, wherein the Ang-(1-7) oligopeptide derivative is administered to the subject for a period of from about 1 month to about 1 year.

44. The method of any one of claims 1-43, wherein the subject is a human subject.

45. The method of claim 44, wherein the subject has been diagnosed as having Alzheimer's disease.


SEQUENCE LISTING (13 sequences)

SEQ ID NO: 1 - skipped

SEQ ID NO: 2 - protein, 7 aa, synthetic construct
DRVYIHP

SEQ ID NO: 3 - protein, 7 aa, synthetic construct
  SITE 1: note = D-Asp
  SITE 2: note = D-Arg
  SITE 3: note = D-Val
  SITE 4: note = D-Tyr
  SITE 5: note = D-Ile
  SITE 6: note = D-His
  SITE 7: note = D-Pro
DRVYIHP

SEQ ID NO: 4 - protein, 7 aa, synthetic construct
DRVYIHP

SEQ ID NO: 5 - protein, 7 aa, synthetic construct
  SITE 1: note = D-Asp
  SITE 2: note = D-Arg
  SITE 3: note = D-Val
  SITE 4: note = D-Tyr
  SITE 5: note = D-Ile
  SITE 6: note = D-His
  SITE 7: note = D-Pro
DRVYIHP

SEQ ID NO: 6-13 - skipped (8 entries)